(19) United States
(12) Patent Application Publication
(10) Pub. No.: US 2011/0144062 A1
(43) Pub. Date: Jun. 16, 2011

(54) DERMATOLOGICAL ANTI-WRINKLE AGENT

(75) Inventors: Eiko KATO, Chiba-shi (JP); Toshi TSUZUKI, Chiba-shi (JP)

(73) Assignee: SHOWA DENKO K.K., Tokyo (JP)

(21) Appl. No.: 13/032,847

(22) Filed: Feb. 23, 2011

Related U.S. Application Data
(62) Division of application No. 12/306,549, filed on Dec. 24, 2008.

(30) Foreign Application Priority Data
Jun. 27, 2006 (JP) 2006-177163

(51) Int. Cl. A61K 8/55; A61Q 19/08

(52) U.S. Cl. 514/100

(57) ABSTRACT

A dermatological anti-wrinkle agent and an anti-wrinkle cosmetic are stable and satisfactorily effective against wrinkles. The dermatological anti-wrinkle agent includes a tocopherol phosphate represented by the following formula (1):

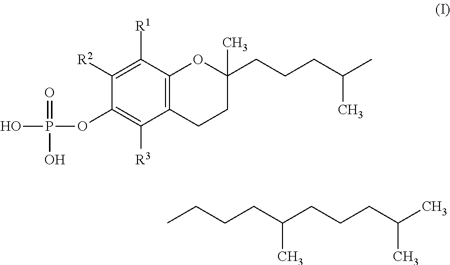

and/or a salt thereof, wherein R1, R2, and R3 represent a hydrogen atom or a methyl group. The anti-wrinkle cosmetic includes the anti-wrinkle agent.

### CROSS REFERENCE TO RELATED APPLICATIONS

This is a divisional of application Ser. No. 12/306,549 filed Dec. 24, 2008, which is the National Stage of PCT/JP2007/063187 filed Jun. 25, 2007, the disclosures of which are hereby incorporated by reference.

### FIELD OF THE INVENTION

The present invention relates to a dermatological anti-wrinkle agent which contains a tocopherol phosphate represented by the following formula (1):

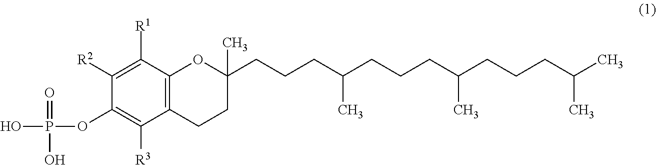

and/or a salt thereof, wherein R1, R2, and R3 represent a hydrogen atom or a methyl group independent from each other.

In the present specification, the term anti-wrinkle means general effects for suppressing the formation and retention of wrinkles, such as prevention of wrinkle formation and treatment of already formed wrinkles.

### BACKGROUND OF THE INVENTION

Skin is always exposed to external stimuli. Consequently, wrinkles, flabbiness, dullness and pigmentation occur in the skin with age, and the properties of the skin change. This is called skin aging. The mechanism of aging is on the way of clarification and not yet cleared. The skin is the outermost layer of a living body to play a protective role. Therefore, it is thought that the accumulation of damages caused by environmental factors is largely responsible for the skin aging. In particular, it is thought that an ultraviolet light is the most serious environmental factor.

It is known that free radicals and active oxygen that are produced by an ultraviolet light induce not only acute inflammation such as a sunburn but also photoaging resulting from chronic exposure. It is thought that the free radicals and active oxygen generated by an ultraviolet light will damage and denature the crosslinks of elastic protein such as collagen and elastin which is a constituent of a corium, will inactivate the antioxidant enzymes such as SOD, and will cause the peroxidation of cell membrane lipid, thereby resulting in wrinkles.

Conventionally, dermatological anti-wrinkle agents and cosmetics are proposed for the purposes of preventing and improving the deformation of skin due to aging. Patent documents 1 to 5 disclose agents including tocopherol or an oil soluble tocopherol derivative such as tocopherol acetate. However, the stability of the tocopherol and the oil soluble tocopherol derivative is insufficient and consequently the dermatological agents including them are poorly stable. Consequently, it is difficult that the agents contain such compounds in amounts enough to obtain wrinkle prevention effects.

Moreover, dermatological agents and cosmetics including a water soluble tocopherol derivative such as tocopherol phosphate have been proposed (see Patent documents 6 and 7). However, wrinkle prevention effects thereof have not been reported.

Furthermore, dermatological agents including a water soluble tocopherol derivative and a collagen peptide have been proposed (see Patent document 8). However, wrinkle prevention effects obtained by the water soluble tocopherol derivative alone or in combination with an ascorbic acid derivative have not been reported.
Patent document 1: Japanese Patent Application Laid-Open Publication No. 62-19511
Patent document 2: Japanese Patent Application Laid-Open Publication No. 62-87506
Patent document 3: Japanese Patent Application Laid-Open Publication No. 8-225428
Patent document 4: Japanese Patent Application Laid-Open Publication No. 2004-168702
Patent document 5: Japanese Patent Application Laid-Open Publication No. 2005-112742
Patent document 6: Japanese Patent Application Laid-Open Publication No. 4-208209
Patent document 7: Japanese Patent Application Laid-Open Publication No. 2004-26817
Patent document 8: Japanese Patent Application Laid-Open Publication No. 2006-143660

### DISCLOSURE OF THE INVENTION

The present invention has been made in consideration of such conditions, and an object of the present invention is to provide a dermatological anti-wrinkle agent and an anti-wrinkle cosmetic containing the anti-wrinkle agent, which are both stable and satisfactorily effective against wrinkles.

The present inventors have diligently studied in order to solve the above problems of the conventional art. The inventors have found that a dermatological anti-wrinkle agent and an anti-wrinkle cosmetic which are highly stable and excellent in wrinkle prevention effects can be obtained by compounding a tocopherol phosphate as an active ingredient. The inventors have completed the present invention based on the finding. More specifically, the present invention includes the following items.

(1) A dermatological anti-wrinkle agent comprising a tocopherol phosphate represented by the following formula (1):

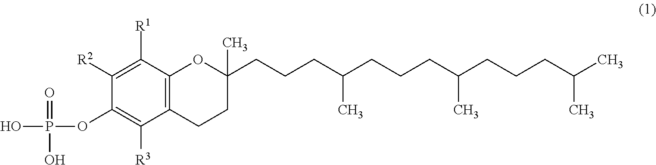

and/or a salt thereof, wherein R1, R2, and R3 represent a hydrogen atom or a methyl group independent from each other.

(2) The dermatological anti-wrinkle agent as defined in above (1), wherein the tocopherol phosphate is α-tocopherol phosphate.

(3) The dermatological anti-wrinkle agent as defined in above (2), wherein the tocopherol phosphate is dl-α-tocopherol phosphate.

(4) The dermatological anti-wrinkle agent as defined in above (2), wherein the tocopherol phosphate is d-α-tocopherol phosphate.

(5) The dermatological anti-wrinkle agent as defined in above (1), wherein the tocopherol phosphate is γ-tocopherol phosphate.

(6) The dermatological anti-wrinkle agent as defined in above (5), wherein the tocopherol phosphate is d-γ-tocopherol phosphate.

(7) The dermatological anti-wrinkle agent as defined in above (1), wherein the tocopherol phosphate is δ-tocopherol phosphate.

(8) The dermatological anti-wrinkle agent as defined in above (7), wherein the tocopherol phosphate is d-δ-tocopherol phosphate.

(9) The dermatological anti-wrinkle agent as defined in any one of above (1) to (8), wherein the salt of the tocopherol phosphate is an alkali metal salt of the tocopherol phosphate.

(10) The dermatological anti-wrinkle agent as defined in above (9), wherein the alkali metal salt of the tocopherol phosphate is sodium salt of the tocopherol phosphate.

(11) The dermatological anti-wrinkle agent as defined in any one of above (1) to (10), wherein the percentage content of the tocopherol phosphate and/or the salt thereof is in the range of 0.01 to 20 mass %.

(12) The dermatological anti-wrinkle agent as defined in any one of above (1) to (11), wherein the agent further comprises an ascorbic acid derivative in addition to the tocopherol phosphate and/or the salt thereof.

(13) The dermatological anti-wrinkle agent as defined in above (12), wherein the ascorbic acid derivative is at least one compound selected from ascorbic acid-2-phosphate, ascorbic acid-2-glucoside, ascorbic acid-6-palmitate, ascorbic acid-2-phosphate-6-palmitate, ascorbic acid-2-phosphate-6-hexyldecanoate, ascorbic acid-2-phosphate-6-stearate, and salts thereof.

(14) The dermatological anti-wrinkle agent as defined in above (12) or (13), wherein the percentage content of the ascorbic acid derivative is in the range of 0.03 to 20 mass %.

(15) An anti-wrinkle cosmetic comprising the dermatological anti-wrinkle agent as defined in any one of above (1) to (14).

The dermatological anti-wrinkle agent of the present invention has an extremely stable property and satisfactory wrinkle prevention effects, and can be widely used for the purpose of wrinkle prevention.

Moreover, the dermatological anti-wrinkle agent in accordance with the present invention has satisfactory wrinkle prevention effects, that is to say, satisfactory effects for stimulating the collagen synthesis and for suppressing the collagen decomposition, and can prevent and improve the deformation of skin due to aging. Consequently, the dermatological anti-wrinkle agent can be widely applied to pharmaceuticals, semi-pharmaceuticals, cosmetics, etc.

### BEST MODE FOR CARRYING OUT THE INVENTION

The present invention will be described below in detail.

The dermatological anti-wrinkle agent in accordance with the present invention includes a tocopherol phosphate represented by the following formula (1):

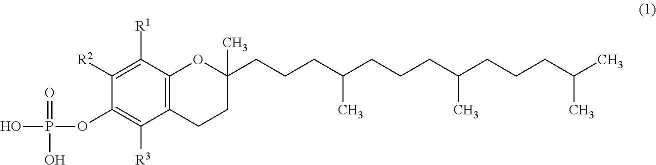

and/or a salt thereof, wherein R1, R2, and R3 represent a hydrogen atom or a methyl group independent from each other.

Of the tocopherol phosphates represented by the above formula (1), typical and preferred examples include α-tocopherol phosphate wherein R1, R2, and R3 are methyl groups; γ-tocopherol phosphate wherein R1 and R2 are methyl groups and R3 is a hydrogen atom; and δ-tocopherol phosphate wherein R1 is a methyl group and R2 and R3 are hydrogen atoms.

The tocopherol phosphates represented by the above formula (1) contain an asymmetric carbon atom in the 2-position of the chroman ring. Consequently, d stereoisomers, l stereoisomers and dl isomers are possible in the tocopherol phosphates. The present invention may use any of these isomers.

Preferred examples of the salts of the tocopherol phosphates include alkali metal salts such as sodium salt and potassium salt, alkaline earth metal salts such as magnesium salt and calcium salt, ammonium salts, alkylammonium salts, and zinc salts of the tocopherol phosphates.

Among them, the alkali metal salts of the tocopherol phosphates, particularly sodium salt, are preferable because they have high solubility in water and their powder shape permits easy handling.

The tocopherol phosphate and/or the salt thereof may be obtained by publicly known methods such as the method described in Japanese Patent Application Laid-Open Publication No. 59-44375 or WO97/14705.

For instance, tocopherol dissolved in a solvent may be reacted with a phosphorylating agent such as phosphorus oxychloride and may be thereafter purified appropriately to produce a tocopherol phosphate.

The thus-obtained tocopherol phosphate may be neutralized with a metal oxide such as magnesium oxide, a metal hydroxide such as sodium hydroxide, ammonium hydroxide, or alkylammonium hydroxide, and thereby a salt of the tocopherol phosphate may be easily obtained.

The tocopherol phosphate and/or the salt thereof accounts for 0.01 to 20 mass %, preferably 0.03 to 20 mass %, more preferably 0.05 to 12 mass % of the total amount of the dermatological anti-wrinkle agent. This percentage content applies irrespective of whether the tocopherol phosphate or the salt thereof is used singly or they are used in combination.

In the case in which the percentage content of the tocopherol phosphate and/or the salt thereof in the dermatological anti-wrinkle agent is in the above range, the active ingredient (s) can produce sufficient wrinkle prevention effects, the preparation of the agent is easy, and the stability of the agent is easily maintained.

Moreover, the dermatological anti-wrinkle agent in accordance with the present invention may further contain an ascorbic acid derivative in addition to the tocopherol phosphate and/or the salt thereof. The use of the ascorbic acid derivative improves wrinkle prevention effects.

The ascorbic acid derivative used in the present invention may be any derivative that liberates ascorbic acid in a living body. In the present specification, the ascorbic acid and ascorbic acid derivative represent L-ascorbic acid and L-ascorbic acid derivative, respectively.

Examples of the ascorbic acid derivatives include ascorbic acid-2-phosphate, ascorbic acid-2-glucoside, ascorbic acid-6-palmitate, ascorbic acid dipalmitate, ascorbic acid-2-phosphate-6-palmitate, ascorbic acid-2-phosphate-6-hexyldecanoate, ascorbic acid-6-stearate, ascorbic acid-2-phosphate-6-stearate, ascorbic acid-6-tetraisopalmitate, ascorbic acid sulfate, and salts thereof. Among them, preferable are ascorbic acid-2-phosphate, ascorbic acid-2-glucoside, ascorbic acid-6-palmitate, ascorbic acid-2-phosphate-6-palmitate, ascorbic acid-2-phosphate-6-hexyldecanoate, ascorbic acid-2-phosphate-6-stearate, and salts thereof. Among them, more preferable are ascorbic acid-2-phosphate-6-palmitate, ascorbic acid-2-phosphate-6-hexyldecanoate, and salts thereof.

Examples of the salts of the ascorbic acid derivatives include alkali metal salts and alkaline earth metal salts of the above-mentioned compounds.

The ascorbic acid derivative may be used in an amount which is pharmaceutically acceptable and which is such that sufficient ascorbic acid will be liberated enough to reduce tocopherol liberated from the tocopherol phosphate and/or the salt thereof in a living body. The percentage content of the ascorbic acid derivative is usually in the range of 0.03 to 20 mass %, preferably 0.05 to 12 mass %, more preferably 0.1 to 10 mass % in the dermatological anti-wrinkle agent.

To make it sure that the ascorbic acid derivative will liberate ascorbic acid enough to reduce tocopherol liberated from the tocopherol phosphate and/or the salt thereof in a living body, the ascorbic acid derivative is more preferably used in an amount of 0.5 to 3 parts by mass per 1 part by mass of the tocopherol phosphate and/or the salt thereof.

In addition to the tocopherol phosphate and/or the salt thereof and the ascorbic acid derivative, the dermatological anti-wrinkle agent and the anti-wrinkle cosmetic of the present invention may contain general dermatological and cosmetic components as required while still achieving the effects of the invention.

Examples of such components include:

hydrocarbons such as ozokerite, α-olefin oligomers, light isoparaffin, light liquid isoparaff in, squalene, squalane, synthetic squalane, vegetable squalane, ceresin, paraffin, polyethylene powder, polybutene, microcrystalline wax, liquid isoparaffin, liquid paraffin, mineral oil and vaseline;

natural waxes such as jojoba oil, carnauba wax, candelilla wax, rice bran wax, shellac, lanolin, mink oil wax, whale wax, sugarcane wax, sperm oil, beeswax and montan wax;

natural fats and oils such as avocado oil, almond oil, olive oil, extra virgin olive oil, sesame oil, rice bran oil, rice oil, rice germ oil, corn oil, soybean oil, maize oil, persic oil, palm kernel oil, palm oil, castor oil, grape seed oil, cotton seed oil, coconut oil, hydrogenated coconut oil, beef tallow, hydrogenated oil, horse oil, mink oil, egg yolk oil, egg yolk fatty oil, rose hip oil, kukui nut oil, evening primrose oil, wheat germ oil, peanut oil, camellia oil, sasanqua oil, cacao butter, Japanese wax, beef bone fat, neatsfoot oil, lard, horse fat, mutton tallow, shea butter, macadamia nut oil and meadowfoam oil;

fatty acids such as lauric acid, myristic acid, palmitic acid, stearic acid, behenic acid, oleic acid, isostearic acid, 12-hydroxystearic acid, undecylenic acid and coconut fatty acid;

higher alcohols such as isostearyl alcohol, octyldodecanol, hexyldecanol, cholesterol, phytosterol, lauryl alcohol, myristyl alcohol, cetanol, stearyl alcohol, oleyl alcohol, behenyl alcohol and cetostearyl alcohol;

alkyl glyceryl ethers such as batyl alcohol, chimyl alcohol, selachyl alcohol and isostearyl glyceryl ether;

esters such as isopropyl myristate, butyl myristate, isopropyl palmitate, ethyl stearate, butyl stearate, ethyl oleate, ethyl linoleate, isopropyl linoleate, cetyl caprylate, hexyl laurate, isooctyl myristate, decyl myristate, myristyl myristate, cetyl myristate, octadecyl myristate, cetyl palmitate, stearyl stearate, decyl oleate, oleyl oleate, cetyl ricinoleate, isostearyl laurate, isotridecyl myristate, isocetyl myristate, isostearyl myristate, octyldodecyl myristate, 2-ethylhexyl palmitate, isocetyl palmitate, isostearyl palmitate, 2-ethylhexyl stearate, isocetyl stearate, isodecyl oleate, octyldodecyl oleate, octyldodecyl ricinoleate, ethyl isostearate, isopropyl isostearate,

cetyl 2-ethylhexanoate, cetostearyl 2-ethylhexanoate, stearyl 2-ethylhexanoate, hexyl isostearate, ethylene glycol dioctanoate, ethylene glycol dioleate, propylene glycol dicaprylate, propylene glycol di(caprylate caprate), propylene glycol dicaprate, propylene glycol dioleate, neopentyl glycol dicaprate, neopentyl glycol dioctanoate, glyceryl tricaprylate, glyceryl tri-2-ethylhexanoate, glyceryl tri(caprylate caprate), glyceryl tri(caprylate caprate stearate), glyceryl triundecylate, glyceryl triisopalmitate, glyceryl triisostearate, trimethylolpropane tri-2-ethylhexanoate, trimethylolpropane triisostearate, pentaerythrityl tetra-2-ethylhexanoate,

pentaerythrityl tetramyristate, pentaerythrityl tetraisostearate, diglyceryl tetraisostearate, octyldodecyl neopentanoate, isocetyl octanoate, isostearyl octanoate, 2-ethylhexyl isopelargonate, hexyldecyl dimethyloctanoate, octyldodecyl dimethyloctanoate, 2-ethylhexyl isopalmitate, isocetyl isostearate, isostearyl isostearate, octyldodecyl isostearate, lauryl lactate, myristyl lactate, cetyl lactate, octyldodecyl lactate, triethyl citrate, acetyltriethyl citrate, acetyltributyl citrate, trioctyl citrate, triisocetyl citrate, trioctyldodecyl citrate, diisostearyl malate, 2-ethylhexyl hydroxystearate, di-2-ethylhexyl succinate, diisopropyl adipate, diisobutyl adipate,

dioctyl adipate, diheptylundecyl adipate, diethyl sebacate, diisopropyl sebacate, dioctyl sebacate, cholesteryl stearate, cholesteryl isostearate, cholesteryl hydroxystearate, cholesteryl oleate, dihydrocholesteryl oleate, phytosteryl isostearate, phytosteryl oleate, isocetyl 12-stearoylhydroxystearate, stearyl 12-stearoylhydroxystearate, isostearyl 12-stearoylhydroxystearate, polyoxyethylene (3) polyoxypropylene (1) cetyl ether acetate, polyoxyethylene (3) polyoxypropylene (1) isocetyl ether acetate, isononyl isononanoate, octyl isononanoate, tridecyl isononanoate and isotridecyl isononanoate;

silicone oils such as methyl polysiloxane, methylphenyl polysiloxane, methylhydrogen polysiloxane, methyl cyclopolysiloxane, octamethyl cyclotetrasiloxane, decamethyl cyclopentasiloxane, dodecamethyl cyclohexasiloxane, octamethyl trisiloxane, decamethyl tetrasiloxane, tetradecamethyl hexasiloxane, highly polymerized methyl polysiloxane, dimethyl siloxane/methyl(polyoxyethylene)siloxane/methyl(polyoxypropylene)siloxane copolymer, dimethyl siloxane/methyl(polyoxyethylene)siloxane copolymer, dimethyl siloxane/methyl(polyoxypropylene)siloxane copolymer, dimethyl siloxane/methyl cetyloxysiloxane copolymer, dimethyl siloxane/methyl stearoxysiloxane copolymer, polyether-modified silicones, alcohol-modified silicones, alkyl-modified silicones and amino-modified silicones;

polymers such as sodium alginate, carrageenan, agar, furcelleran, cyamoposis gum, pyrus cyclonia seed, konjac mannan, tamarind gum, tara gum, dextrin, starch, locust bean gum, gum arabic, ghatti gum, karaya gum, tragacanth gum, arabinogalactan, pectin, marmelo, chitosan, curdlan, xanthan gum, gellan gum, cyclodextrin, dextran, pullulan, microcrystalline cellulose, methylcellulose, ethylcellulose, hydroxyethylcellulose, hydroxypropylcellulose, hydroxypropylmethylcellulose, carboxymethylcellulose, carboxy starch, cationized cellulose, starch phosphate, cationized cyamoposis gum, carboxymethyl/hydroxypropylated cyamoposis gum, hydroxypropylated cyamoposis gum, albumin, casein, gelatin,

sodium polyacrylate, polyacrylic acid amide, carboxyvinyl polymers, polyethyleneimine, highly polymerized polyethylene glycol, polyvinyl alcohol, polyvinylpyrrolidone, polyvinyl ether, polyacrylamide, acrylic acid copolymers, methacrylic acid copolymers, maleic acid copolymers, vinylpyridine copolymers, ethylene/acrylic acid copolymers, vinylpyrrolidone polymers, vinyl alcohol/vinylpyrrolidone copolymers, nitrogen-substituted acrylamide polymers, amino-modified silicones, cationized polymers, dimethylacryl ammonium polymers, acrylic acid-based anionic polymers, methacrylic acid-based anionic polymers, modified silicones, alkyl (C₁₀₋₃₀) acrylate or methacrylate copolymers and polyoxyethylene/polyoxypropylene copolymer;

lower alcohols such as ethanol, isopropyl alcohol, 1-butanol, 2-butanol and benzyl alcohol;

polyhydric alcohols such as ethylene glycol, diethylene glycol, polyethylene glycol, propylene glycol, polypropylene glycol, glycerol, diglycerol, polyglycerol, 1,3-butanediol, triethylene glycol, dipropylene glycol, 3-methyl-1,3-butanediol, 1,2-pentanediol, 1,4-pentanediol, 1,5-pentanediol, 2,4-pentanediol, 2-methyl-2,4-pentanediol, 3-methyl-1,5-pentanediol, 1,2-hexanediol and 1,6-hexanediol;

anionic surfactants such as potassium coconut fatty acid ester, sodium coconut fatty acid ester, triethanolamine coconut fatty acid ester, potassium laurate, sodium laurate, triethanolamine laurate, potassium myristate, sodium myristate, isopropanolamine myristate, potassium palmitate, sodium palmitate, isopropanolamine palmitate, potassium stearate, sodium stearate, triethanolamine stearate, potassium oleate, sodium oleate, sodium castor oil fatty acid ester, zinc undecylenate, zinc laurate, zinc myristate, magnesium myristate, zinc palmitate, zinc stearate, calcium stearate, magnesium stearate, aluminum stearate, calcium myristate, magnesium myristate, aluminum dimyristate, aluminum isostearate,

polyoxyethylene laurylether acetic acid, sodium polyoxyethylene laurylether acetate, polyoxyethylene tridecylether acetic acid, sodium polyoxyethylene tridecylether acetate, sodium stearoyl lactate, sodium isostearoyl lactate, lauroylsarcosine sodium, sarcosine coconut fatty acid ester, sarcosine sodium coconut fatty acid ester, sarcosine triethanolamine coconut fatty acid ester, lauroyl sarcosine, lauroyl sarcosine potassium, lauroylsarcosine triethanolamine, oleoyl sarcosine, myristoyl sarcosine sodium, sodium stearoyl glutamate, coconut fatty acid acylglutamic acid, coconut fatty acid potassium acylglutamate, coconut fatty acid sodium acylglutamate, coconut fatty acid triethanolamine acylglutamate, lauroyl acylglutamic acid, potassium lauroyl acylglutamate,

sodium lauroyl acylglutamate, triethanolamine lauroyl acylglutamate, myristoyl acylglutamic acid, potassium myristoyl acylglutamate, sodium myristoyl acylglutamate, stearoyl acylglutamic acid, potassium stearoyl acylglutamate, disodium stearoyl acylglutamate, hydrogenated tallow fatty acid sodium acylglutamate, coconut fatty acid/hydrogenated tallow fatty acid sodium acylglutamate, methylalanine sodium coconut fatty acid ester, lauroyl methylalanine, lauroyl methylalanine sodium, lauroyl methylalanine triethanolamine, myristoyl methylalanine sodium, lauroyl methyltaurine sodium, methyltaurine potassium coconut fatty acid ester, methyltaurine sodium coconut fatty acid ester, methyltaurine magnesium coconut fatty acid ester, myristoyl methyltaurine sodium,

palmitoyl methyltaurine sodium, stearoyl methyltaurine sodium, oleoyl methyltaurine sodium, sodium alkanesulfonate, sodium tetradecenesulfonate, dioctylsodium sulfosuccinate, lauryl disodium sulfosuccinate, ethyl coconut fatty acid ester sodium sulfonate, sodium laurylsulfate, triethanolamine laurylsulfate, sodium cetyl sulfate, triethanolamine alkyl (11, 13, 15) sulfates, sodium alkyl (12, 13) sulfates, triethanolamine alkyl (12, 13) sulfates, ammonium alkyl (12, 14, 16) sulfates, diethanolamine alkyl (12, 13) sulfates, triethanolamine alkyl (12-14) sulfates, triethanolamine alkyl (12-15) sulfates, magnesium triethanolamine cocoalkylsulfate, ammonium laurylsulfate, potassium laurylsulfate,

magnesium laurylsulfate, monoethanolamine laurylsulfate, diethanolamine laurylsulfate, sodium myristylsulfate, sodium stearylsulfate, sodium oleylsulfate, triethanolamine oleylsulfate, sodium polyoxyethylene laurylether sulfate, triethanolamine polyoxyethylene laurylether sulfate, sodium polyoxyethylene (1) alkyl (11, 13, 15) ether sulfate, triethanolamine polyoxyethylene (1) alkyl (11, 13, 15) ether sulfate, sodium polyoxyethylene (3) alkyl (11-15) ether sulfate, sodium polyoxyethylene (2) alkyl (12, 13) ether sulfate, sodium polyoxyethylene (3) alkyl (12-14) ether sulfate, sodium polyoxyethylene (3) alkyl (12-15) ether sulfate, sodium polyoxyethylene (2) laurylether sulfate,

sodium polyoxyethylene (3) myristylether sulfate, higher fatty acid alkanolamide sulfate sodium, laurylphosphoric acid, sodium laurylphosphate, potassium cetylphosphate, diethanolamine cetylphosphate, polyoxyethylene oleylether phosphoric acid, polyoxyethylene laurylether phosphoric acid, sodium polyoxyethylene laurylether phosphate, polyoxyethylene cetylether phosphoric acid, sodium polyoxyethylene cetylether phosphate, polyoxyethylene stearylether phosphoric acid, polyoxyethylene oleylether phosphoric acid, sodium polyoxyethylene oleylether phosphate, polyoxyethylene alkylphenyl ether phosphoric acid, sodium polyoxyethylene alkylphenyl ether phosphate, triethanolamine polyoxyethylene alkylphenyl ether phosphate, polyoxyethylene octylether phosphoric acid, polyoxyethylene (10) alkyl (12, 13) ether phosphoric acid, polyoxyethylene alkyl (12-15) ether phosphoric acid, polyoxyethylene alkyl (12-16) ether phosphoric acid, triethanolamine polyoxyethylene laurylether phosphate and diethanolamine polyoxyethylene oleylether phosphate;

cationic surfactants such as dioctylamine, dimethylstearylamine, trilaurylamine, stearic acid diethylaminoethylamide, lauryltrimethylammonium chloride, cetyltrimethylammonium chloride, cetyltrimethylammonium bromide, cetyltrimethylammonium saccharin, stearyltrimethylammonium chloride, alkyl (20-22) trimethylammonium chloride, lauryltrimethylammonium bromide, alkyl (16, 18) trimethylammonium chloride, stearyltrimethylammonium bromide, stearyltrimethylammonium saccharin, alkyl (28) trimethylammonium chloride, di(polyoxyethylene)oleylmethylammonium chloride (2EO), dipolyoxyethylenestearylmethylammonium chloride, polyoxyethylene (1) polyoxypropylene (25) diethylmethylammonium chloride,

tri(polyoxyethylene)stearylammonium chloride (5EO), distearyldimethylammonium chloride, dialkyl (12-15) dimethylammonium chloride, dialkyl (12-18) dimethylammonium chloride, dialkyl (14-18) dimethylammonium chloride, dicocoyldimethylammonium chloride, dicetyldimethylammonium chloride, isostearyllauryldimethylammonium chloride, benzalkonium chloride, myristyldimethylbenzylammonium chloride, lauryldimethyl(ethylbenzyl)ammonium chloride, stearyldimethylbenzylammonium chloride, laurylpyridinium chloride, cetylpyridinium chloride, lauroylcolaminoformylmethylpyridinium chloride, stearoylcolaminoformylmethylpyridinium chloride, alkylisoquinolium bromide, methylbenzethonium chloride and benzethonium chloride;

amphoteric surfactants such as 2-alkyl-N-carboxymethyl-N-hydroxyethyl imidazolinium betaine, alkyldiaminoethylglycine hydrochloride, lauryldiaminoethylglycine sodium, undecylhydroxyethylimidazolium betaine sodium, undecyl-N-carboxymethylimidazolium betaine, acyl-N-carboxyethyl-N-hydroxyethylethylenediamine disodium coconut fatty acid ester, acyl-N-carboxyethoxyethyl-N-carboxyethylethylenediamine disodium coconut fatty acid ester, acyl-N-carboxymethoxyethyl-N-carboxymethylethylenediamine disodium coconut fatty acid ester, sodium laurylaminopropionate, sodium laurylaminodipropionate, triethanolamine laurylaminopropionate, acyl-N-carboxyethyl-N-hydroxyethylethylenediamine sodium palm oil fatty acid ester, betaine lauryldimethylaminoacetate,

betaine coconut oil alkyldimethylaminoacetate, betaine stearyldimethylaminoacetate, stearyldimethyl betaine sodium, amidopropylbetaine coconut fatty acid ester, amidopropylbetaine palm oil fatty acid ester, lauric acid amide betaine propylacetate, ricinoleic acid amide propylbetaine, stearyldihydroxyethyl betaine and laurylhydroxysulfobetaine;

nonionic surfactants such as polyoxyethylene (10) alkyl (12, 13) ether, polyoxyethylene lauryl ether, polyoxyethylene cetyl ether, polyoxyethylene stearyl ether, polyoxyethylene oleyl ether, polyoxyethylene (3, 7, 12) alkyl (12-14) ether, polyoxyethylene tridecyl ether, polyoxyethylene myristyl ether, polyoxyethylene-sec-alkyl (14) ether, polyoxyethylene isocetyl ether, polyoxyethylene cetostearyl ether, polyoxyethylene (2, 10, 20) isostearyl ether, polyoxyethylene oleylcetyl ether, polyoxyethylene (20) arachyl ether, polyoxyethylene octyldodecyl ether, polyoxyethylene behenyl ether, polyoxyethylene octylphenyl ether,

polyoxyethylene nonylphenyl ether, polyoxyethylene dinonylphenyl ether, polyoxyethylene (1) polyoxypropylene (1, 2, 4, 8) cetyl ether, polyoxyethylene (5) polyoxypropylene (1, 2, 4, 8) cetyl ether, polyoxyethylene (10) polyoxypropylene (1, 2, 4, 8) cetyl ether, polyoxyethylene (20) polyoxypropylene (1, 2, 4, 8) cetyl ether, polyoxyethylene polyoxypropylene lauryl ether, polyoxyethylene (3) polyoxypropylene (34) stearyl ether, polyoxyethylene (4) polyoxypropylene (30) stearyl ether, polyoxyethylene (34) polyoxypropylene (23) stearyl ether, polyoxyethylene polyoxypropylene cetyl ether, polyoxyethylene polyoxypropylene decyltetradecyl ether,

polyethylene glycol monolaurate, ethylene glycol monostearate, polyethylene glycol monostearate, polyethylene glycol monooleate, ethylene glycol fatty acid ester, self-emulsifiable ethylene glycol monostearate, diethylene glycol laurate, polyethylene glycol myristate, polyethylene glycol palmitate, diethylene glycol stearate, self-emulsifiable polyethylene glycol (2) monostearate, polyethylene glycol isostearate, ethylene glycol dioctanoate, diethylene glycol dilaurate, polyethylene glycol dilaurate, polyethylene glycol (150) dipalmitate, ethylene glycol distearate, diethylene glycol distearate, polyethylene glycol distearate, ethylene glycol dioleate, polyethylene glycol dioleate,

polyethylene glycol diricinoleate, polyoxyethylene (20) sorbitan monolaurate, polyoxyethylene (20) sorbitan monopalmitate, polyoxyethylene (6) sorbitan monostearate, polyoxyethylene (20) sorbitan monostearate, polyoxyethylene (20) sorbitan tristearate, polyoxyethylene (6) sorbitan monooleate, polyoxyethylene (20) sorbitan monooleate, polyoxyethylene (20) sorbitan trioleate, sorbitan polyoxyethylene (20) coconut fatty acid ester, polyoxyethylene (10-80) sorbitan monolaurate, polyoxyethylene sorbitan tristearate, polyoxyethylene (20) sorbitan isostearate, polyoxyethylene (150) sorbitan tristearate, polyoxyethylene castor oil, polyoxyethylene hydrogenated castor oil,

polyoxyethylene (10) hydrogenated castor oil, polyoxyethylene (20) hydrogenated castor oil, polyoxyethylene (40) hydrogenated castor oil, polyoxyethylene (50) hydrogenated castor oil, polyoxyethylene (60) hydrogenated castor oil, lipophilic glyceryl monostearate, lipophilic glyceryl monooleate, self-emulsifiable glyceryl monostearate, glyceryl coconut fatty acid ester, glyceryl laurate, glyceryl myristate, glyceryl isostearate, glyceryl ricinoleate, glyceryl monohydroxystearate, glyceryl oleate, glyceryl linoleate, glyceryl erucate, glyceryl behenate, wheat germ fatty acid glyceride, glyceryl safflower oil fatty acid ester, glyceryl hydrogenated soybean fatty acid ester, saturated fatty acid glyceride, glyceryl cotton seed oil fatty acid, monoisostearic acid glyceryl monomyristate, monotallow fatty acid glyceride, monoglyceryl lanolin fatty acid ester, glyceryl sesquioleate, glyceryl distearate, glyceryl diisostearate,

glyceryl diarachidate, sorbitan monolaurate, sorbitan monopalmitate, sorbitan monostearate, sorbitan monoisostearate, sorbitan monooleate, sorbitan sesquistearate, sorbitan sesquioleate, sorbitan tristearate, sorbitan trioleate, sorbitan coconut fatty acid ester, sorbitanisostearate, sorbitan sesquiisostearate, sorbitan distearate, diglyceryl isopalmitate, poly (4-10) glyceryl monolaurate, poly (10) glyceryl monomyristate, poly (2-10) glyceryl monostearate, poly (2-10) glyceryl monoisostearate, poly (2-10) glyceryl monooleate, diglyceryl sesquioleate, poly (2-10) glyceryl diisostearate, poly (6-10) glyceryl distearate,

diglyceryl triisostearate, poly (10) glyceryl tristearate, poly (10) glyceryl trioleate, poly (2) glyceryl tetraisostearate, decaglyceryl pentastearate, poly (6-10) glyceryl pentaoleate, poly (10) glyceryl heptastearate, decaglyceryl decastearate, poly (10) glyceryl decaoleate, condensed poly (6) glyceryl ricinoleate, sucrose fatty acid ester, sucrose coconut fatty acid ester, alkyl glucoside, coconut oil alkyldimethylamine oxide, lauryldimethylamine oxide, dihydroxyethyllauryldimethylamine oxide, stearyldimethylamine oxide, oleyldimethylamine oxide and polyoxyethylene coconut oil alkyldimethylamine oxide;

natural surfactants such as saponin, lecithin, soybean phospholipid, hydrogenated soybean phospholipid, soybean lysophospholipid, hydrogenated soybean lysophospholipid, egg yolk lecithin, hydrogenated egg yolk lysophosphatidylcholine, phosphatidylcholine, phosphatidylethanolamine, phosphatidylserine, sphingophospholipid, sphingomyelin, ganglioside, bile acid, cholic acid, deoxycholic acid, sodium cholate, sodium deoxycholate, spiculisporic acid, rhamnolipid, trehalose lipid, sophorolipid and mannosylerythritol lipid;

ultraviolet light absorbers, including paraminobenzoic acid derivatives such as paraminobenzoic acid, ethyl paraminobenzoate, glyceryl paraminobenzoate, amyl paradimethylaminobenzoate and 2-ethylhexyl paradimethylaminobenzoate, cinnamic acid derivatives such as benzyl cinnamate, diparamethoxy cinnamic acid glyceryl mono-2-ethylhexanoate, methyl 2,4-diisopropylcinnamate, ethyl 2,4-diisopropylcinnamate, potassium paramethoxycinnamate, sodium paramethoxycinnamate, isopropyl paramethoxycinnamate, 2-ethylhexyl paramethoxycinnamate, 2-ethoxyethyl paramethoxycinnamate and ethyl paraethoxycinnamate, urocanic acid derivatives such as urocanic acid and ethyl urocanate, benzophenone derivatives such as 2,4-dihydroxybenzophenone, 2,2′,4,4′-tetrahydroxybenzophenone, 2-hydroxy-4-methoxy-5-sulfobenzophenonesodium,

2-hydroxy-4-methoxybenzophenone-5-sulfonic acid, 2-hydroxy-4-methoxybenzophenone, 2,2′-dihydroxy-4,4′-dimethoxybenzophenone and 2,2′-dihydroxy-4,4′-dimethoxy-5-sulfobenzophenonesodium, salicylic acid derivatives such as ethylene glycol salicylate, 2-ethylhexyl salicylate, phenyl salicylate, benzyl salicylate, p-tert-butylphenyl salicylate, homomethyl salicylate and 3,3,5-trimethylcyclohexyl salicylate, 2-(2′-hydroxy-5′-methoxyphenyl)benzotriazole and 4-tert-butyl-4′-methoxybenzoylmethane;

powders and color materials such as kaolin, silicic anhydride, aluminum magnesium silicate, sericite, talc, boron nitride, mica, montmorillonite, hemp cellulose powder, wheat starch, silk powder, cornstarch, nitro dye, azo dye, nitroso dye, triphenylmethane dye, xanthene dye, quinoline dye, anthraquinone dye, indigo dye, pyrene dye, phthalocyanine dye, natural dyes including flavonoid, quinone, porphyrin, water-soluble annatto, squid ink powder, caramel, guaiazulene, gardenia blue, gardenia yellow, cochineal, shikonin, copper chlorophyllin sodium, paprika dye, safflower red, safflower yellow, laccaic acid and riboflavin butyrate, carbon black, yellow iron oxide, black iron oxide, red iron oxide, iron blue, ultramarine blue, zinc oxide, chromium oxide, titanium oxide, black titanium oxide, zirconium oxide, chromium hydroxide, alumina, magnesium oxide, barium sulfate, aluminum hydroxide, calcium carbonate, lithium cobalt titanate, manganese violet and pearl pigment;

plant extracts such as angelica extract, gambir extract, avocado extract, hydrangea extract, gynostemma pentaphyllum extract, althea extract, arnica extract, oil-soluble arnica extract, almond extract, aloe extract, styrax resin extract, ginkgo extract, nettle extract, orris extract, fennel extract, turmeric curcuma extract, rose fruit extract, echinacea leaf extract, scutellaria root extract, phellodendron bark extract, Japanese coptis rhizome extract, barley extract, okura extract, hypericum extract, oil-soluble hypericum extract, white nettle extract, oil-soluble white nettle extract, restharrow extract, watercress extract, orange flower water, persimmon tannin, pueraria root extract, Japanese valerian extract, cattail extract, chamomile extract, oil-soluble chamomile extract, chamomile water, oat extract, carrot extract, oil-soluble carrot extract, carrot oil, artemisia capillaris extract, glycyrrhiza extract, glycyrrhiza extracted powder,

glycyrrhiza flavonoid, cantharis tincture, raspberry extract, kiwi extract, cinchona extract, cucumber extract, apricot kernel extract, quince seed extract, gardenia extract, sasa albo-marginata extract, sophora root extract, walnut shell extract, clematis extract, black sugar extract, chlorella extract, mulberry bark extract, cinnamon bark extract, gentian extract, geranium herb extract, black tea extract, nuphar extract, burdock root extract, oil-soluble burdock root extract, wheat germ extract, hydrolyzed wheat powder, rice bran extract, fermented rice bran extract, comfrey extract, asiasarum root extract, saffron extract, saponaria extract, oil-soluble salvia extract, crataegus fruit extract, zanthoxylum fruit extract, shiitake extract, shiitake mushroom extracted powder, rehmannia root extract, lithospermum root extract, oil-soluble lithospermum root extract, perilla herb extract, linden extract, oil-soluble linden extract, filipendula extract, peony root extract, job's tears extract, ginger extract,

oil-soluble ginger extract, ginger tincture, acorus calamus rhizome extract, birch extract, oil-soluble birch extract, birch sap, honeysuckle extract, horsetail extract, oil-soluble horsetail extract, scordinin, stevia extract, ivy extract, crataegus extract, sambucus extract, juniper extract, yarrow extract, oil-soluble yarrow extract, peppermint extract, sage extract, oil-soluble sage extract, sage water, mallow extract, celery extract, cnidium rhizome extract, cnidium rhizome water, swertia herb extract, soy extract, jujube extract, thyme extract, green tea extract, tea leaf dry distillated solution, tee seed extract, clove extract, citrus unshiu peel extract, camellia extract, centella extract, oil-soluble walnut extract, duke extract, terminalia extract, Japanese angelica root extract, oil-soluble Japanese angelica root extract, Japanese angelica root water, calendula extract, oil-soluble calendula extract, soy milk powder, peach seed extract, bitter orange peel extract, houttuynia extract, tomato extract,

tormentilla extract, natto extract, ginseng extract, oil-soluble ginseng extract, garlic extract, wild rose extract, oil-soluble wild rose extract, malt extract, malt root extract, ophiopogon tuber extract, parsley extract, barley leaf juice concentrate, peppermint distillate, witch hazel distillate, witch hazel extract, rose extract, pellitory extract, isodonis extract, loquat leaf extract, oil-soluble loquat leaf extract, coltsfoot extract, hoelen extract, butcher broom extract, butcher broom extracted powder, grape extract, grape leaf extract, grape water, hayflower extract, sponge gourd extract, sponge gourd solution, safflower extract, oil-soluble linden extract, linden water, paeonia extract, hop extract, oil-soluble hop extract, pine extract, silybum marianum fruit extract, horse chestnut extract, oil-soluble horse chestnut extract, mukurossi peel extract, balm mint extract, sweet clover extract, peach leaf extract, oil-soluble peach leaf extract, bean sprouts extract, corn flower extract, corn flower water, eucalyptus extract, saxifraga extract, lily extract, coix extract, oil-soluble coix extract, mugwort extract, Japanese mugwort water, lavender extract, lavender water, apple extract, ganoderma extract, lettuce extract, Chinese milk vetch extract, rose water, rosemary extract, oil-soluble rosemary extract, roman chamomile extract and burnet extract;

amino acids and peptides such as glycine, valine, leucine, isoleucine, serine, threonine, phenylalanine, thyrosin, tryptophan, cystine, cysteine, methionine, hydroxyproline, aspartic acid, asparagine, glutamic acid, glutamine, histidine, γ-aminobutyric acid, DL-pyrrolidonecarboxylic acid, ε-aminocaproic acid, hydrolyzed elastin, water-soluble elastin, hydrolyzed collagen, water-soluble collagen, casein, glutathione, wheat peptide and soybean peptide;

vitamins and vitamin affecters, including vitamin A such as retinol, retinal, retinoic acid, retinol acetate and retinol palmitate, carotenoids such as α-carotene, β-carotene, γ-carotene, δ-carotene, lycopene, zeaxanthin, cryptoxanthin, echinenone and astaxanthin, vitamin B1 such as thiamines, vitamin B2 such as riboflavin, vitamin B6 such as pyridoxine, pyridoxal and pyridoxamine, vitamin B12 such as cyanocobalamin, vitamin C such as folic acids, nicotinic acid, nicotinic acid amide, pantothenic acids, biotins, L-ascorbic acid and ascorbic acid derivatives other than the aforementioned including sodium ascorbate and magnesium ascorbate, vitamin D such as ergocalciferol and cholecalciferol, oil-soluble vitamin E such as d-α-tocopherol, DL-α-tocopherol, dl-α-tocopherol acetate, dl-α-tocopherol succinate, β-tocopherol, γ-tocopherol and d-δ-tocopherol, ubiquinones, vitamin K, carnitine, ferulic acid, γ-oryzanol, α-lipoic acid and orotic acid;

antiseptics such as benzoic acid, sodium benzoate, undecylenic acid, salicylic acid, sorbic acid, potassium sorbate, dehydroacetic acid, sodium dehydroacetate, isobutyl paraoxybenzoate, isopropyl paraoxybenzoate, ethyl paraoxybenzoate, butyl paraoxybenzoate, propyl paraoxybenzoate, benzyl paraoxybenzoate, methyl paraoxybenzoate, methyl sodium paraoxybenzoate, phenoxyethanol, photosensitive agent (kankoh-so) No. 101, photosensitive agent (kankoh-so) No. 201 and photosensitive agent (kankoh-so) No. 401;

antioxidants such as butylhydroxyanisole, butylhydroxytoluene, propyl gallate, erythorbic acid, sodium erythorbate, parahydroxyanisole and octyl gallate;

sequestering agents such as trisodium ethylenediaminehydroxyethyltriacetate, edetic acid, disodium edetate, trisodium edetate, tetrasodium edetate, sodium citrate, gluconic acid, phytic acid, sodium polyphosphate and sodium metaphosphate;

moisturizers such as hyaluronic acid, sodium hyaluronate, sodium chondroitinsulfate, sodium lactate, sodium pyrrolidonecarboxylate, betaine, lactic acid bacteria culture solution, yeast extract and ceramide;

antiinflammatory agents such as glycyrrhizinic acid, trisodium glycyrrhizinate, dipotassium glycyrrhizinate, monoammonium glycyrrhizinate, β-glycyrrhetinic acid, glyceryl glycyrrhetinate, stearyl glycyrrhetinate, lysozyme chloride, hydrocortisone and allantoin;

pH adjusters such as sodium hydroxide, potassium hydroxide and triethanolamine;

salts such as sodium chloride, potassium chloride, magnesium chloride and sodium sulfate;

α-hydroxy acids such as citric acid, glycolic acid, tartaric acid and lactic acid;

whitening agents such as arbutin, α-arbutin and placental extract;

essential oils such as angelica oil, ylang ylang oil, elemi oil, matricaria oil, chamomile oil, cardamom oil, calamus oil, galbanum oil, camphor oil, carrot seed oil, clary sage oil, clove oil, cinnamon bark oil, coriander oil, cypress oil, sandalwood oil, cedarwood oil, citronella oil, cinnamon leaf oil, jasmine absolute, juniper berry oil, ginger extract, spearmint oil, sage oil, cedar oil, geranium oil, thyme oil, tea tree oil, nutmeg oil, niaouli oil, neroli oil, pine oil, basil oil, peppermint oil, patchouli oil, palmarosa oil, fennel oil, petitgrain oil, black pepper oil, frankincense oil, vetivert oil, peppermint oil, bergamot oil, benzoin oil, aniba rosaeodora oil, marjoram oil, myrrh oil, melissa oil, eucalyptus oil, ravensara oil, lavandin oil, lavender oil, lindane oil, rose oil, rosewood oil, rosemary oil and lovage oil;

terpenes such as pinene, terpinene, terpinolene, myrcene and longifolene;

perfumes and water.

In addition to the above-described components, conventional cosmetic materials may also be used as required. For instance, cosmetic materials described in the following references may be used while still achieving the effects of the present invention:
The Japanese Standards of Cosmetic Ingredients, The Second Edition Notes, edited by Society of Japanese Pharmacopoeia, 1984, (YAKUJI NIPPO LIMITED.);
The Japanese Cosmetic Ingredients Codex, supervised by Evaluation and Licensing Division, Pharmaceutical and Medical Safety Bureau, Ministry of Health, Labor and Welfare, 1993, (YAKUJI NIPPO LIMITED.);
Supplement to The Japanese Cosmetic Ingredients Codex, supervised by Evaluation and Licensing Division, Pharmaceutical and Medical Safety Bureau, Ministry of Health, Labor and Welfare, 1993, (YAKUJI NIPPO LIMITED.);
The Comprehensive Licensing Standards of Cosmetics by Category, supervised by Evaluation and Licensing Division, Pharmaceutical and Medical Safety Bureau, Ministry of Health, Labor and Welfare, 1993, (YAKUJI NIPPO LIMITED.);
Compounding Ingredients Codex of Cosmetics by Category, supervised by Evaluation and Licensing Division, Pharmaceutical and Medical Safety Bureau, Ministry of Health, Labor and Welfare, 1997, (YAKUJI NIPPO LIMITED.);
Dictionary of cosmetic materials, 1991, (Nikko Chemicals Co., Ltd.); and
Latest 300 Cosmetic Functional Materials, 2002, (CMC Publishing CO., LTD.).

The dermatological anti-wrinkle agent and the anti-wrinkle cosmetic of the present invention may be formulated in any types or forms that are applicable to skin. Preferably, the formulation is such that the agent or cosmetic can be suitably applied in the vicinity of a region in which wrinkle prevention is desired.

More specifically, the formulations include skin milks, skin creams, foundation creams, massage creams, cleansing creams, shaving creams, cleansing foams, skin lotions, lotions, packs, lipsticks, rouges, eyeshadows, manicures, soaps, body shampoos, hand soaps, shampoos, conditioners, hair tonics, treatments, hair creams, hair sprays, hair restorers, hair-growth medicines, hair dyes, hair liquids, depilatories, dandruff inhibitors, toothpastes, denture adhesives, mouthwashes, permanent waving agents, curling agents, styling agents, ointments, cataplasms, tape agents, bath additives, antiperspirants, and sun blockers. Any formulations applicable to skin may be used. The dermatological anti-wrinkle agent and the anti-wrinkle cosmetic in accordance with the present invention can be used regardless of gender and age of users, and can be applied not only to humans but also to animals.

The formulations may be solids, liquids, semi-solids, gases, fine particles, granules, tablets, gels and foams.

For the tocopherol phosphate represented by the formula (1) and/or the salt thereof to produce effects more effectively, the dermatological anti-wrinkle agent or the anti-wrinkle cosmetic preferably includes an aqueous medium. In this case, the water content is the difference obtained by subtracting the total amount (mass %) of the above components other than water from 100 mass % of the dermatological anti-wrinkle agent or the anti-wrinkle cosmetic. For instance, in the case where the tocopherol phosphate and/or the salt thereof is used in combination with the ascorbic acid derivative, the water content in the dermatological anti-wrinkle agent or the anti-wrinkle cosmetic is generally from more than 0 mass % to 99.96 mass %. When the ascorbic acid derivative is not used, the water content in the dermatological anti-wrinkle agent or the anti-wrinkle cosmetic is generally from more than 0 mass % to 99.99 mass %.

The dermatological anti-wrinkle agent and the anti-wrinkle cosmetic in accordance with the present invention may be manufactured by dissolving, mixing, or dispersing the above components by established methods so as to achieve the predetermined contents, depending on the formulation.

The present invention will be described below by Examples without limiting the scope of the invention.

### EXAMPLES

The amounts in the following tables represent mass unless otherwise specified.

### Examples 1 to 4, Comparative Examples 1 to 4

Creams 1 to 4 (Examples 1 to 4) and creams 5 to 8 (Comparative Examples 1 to 4) were prepared based on the composition ratios listed in the following Table 1. The creams 1 to 8 were prepared by separately heating components A and B listed in Table 1 to 85° C., adding the component B to the component A, emulsifying the components with a homomixer, and cooling the emulsion.

TABLE 1

| | | | | | | Comparative | Comparative | Comparative | Comparative |
|---|---|---|---|---|---|---|---|---|---|
| | | Example 1 | Example 2 | Example 3 | Example 4 | Example 1 | Example 2 | Example 3 | Example 4 |
| | | Cream 1 | Cream 2 | Cream 3 | Cream 4 | Cream 5 | Cream 6 | Cream 7 | Cream 8 |
| A | Hydrogenated rapeseed alcohol | 4.2 | 4.2 | 4.2 | 4.2 | 4.2 | 4.2 | 4.2 | 4.2 |
| | Isononyl isononanoate | 6.0 | 6.0 | 6.0 | 6.0 | 6.0 | 6.0 | 6.0 | 6.0 |
| | Squalane | 9.6 | 9.6 | 9.6 | 9.6 | 9.6 | 9.6 | 9.6 | 9.6 |
| | Octyldodecyl myristate | 4.8 | 4.8 | 4.8 | 4.8 | 4.8 | 4.8 | 4.8 | 4.8 |
| | Polyglyceryl-10 stearate | 2.0 | 2.0 | 2.0 | 2.0 | 2.0 | 2.0 | 2.0 | 2.0 |
| | Glyceryl stearate | 1.0 | 1.0 | 1.0 | 1.0 | 1.0 | 1.0 | 1.0 | 1.0 |
| | Methylparaben | 0.1 | 0.1 | 0.1 | 0.1 | 0.1 | 0.1 | 0.1 | 0.1 |
| | Propylparaben | 0.05 | 0.05 | 0.05 | 0.05 | 0.05 | 0.05 | 0.05 | 0.05 |
| | Xanthan gum | 0.1 | 0.1 | 0.1 | 0.1 | 0.1 | 0.1 | 0.1 | 0.1 |
| | dl-α-tocopherol | — | — | — | — | 2.0 | 0.1 | — | — |
| | Tocopherol acetate | — | — | — | — | — | — | 2.0 | 0.1 |
| B | Tocopherol phosphate sodium salt | 2.0 | 0.1 | 2.0 | 2.0 | — | — | — | — |
| | Ascorbic acid-2-phosphate-6- | — | — | 1.0 | — | — | — | — | — |
| | palmitate trisodium salt | | | | | | | | |
| | Ascorbic acid-2-phosphate-6- | — | — | — | 1.0 | — | — | — | — |
| | hexyldecanoate trisodium salt | | | | | | | | |
| | 1,3-butylene glycol | 4.8 | 4.8 | 4.8 | 4.8 | 4.8 | 4.8 | 4.8 | 4.8 |
| | Glycerin | 4.8 | 4.8 | 4.8 | 4.8 | 4.8 | 4.8 | 4.8 | 4.8 |
| | Trisodium citrate | 0.4 | 0.4 | 0.4 | 0.4 | 0.4 | 0.4 | 0.4 | 0.4 |
| | Purified water | 60.15 | 62.05 | 59.15 | 59.15 | 60.15 | 62.05 | 60.15 | 62.05 |

The numbers are % relative to the total (100%) of the components A and B.

The creams 1 to 8 were each placed into a sample bottle, which was thereafter sealed. The bottles were introduced in a thermostatic chamber at 40° C. and the creams were allowed to stand for 24 weeks. After the passage of 24 weeks, the creams were visually observed to evaluate a change from the state immediately after the cream was placed in the bottle.

The residual tocopherol in the cream after the passage of 24 weeks was analyzed by high performance liquid chromatography under the following conditions. The residual rate was calculated relative to the tocopherol content in the cream immediately after the preparation (100) that had been determined under the similar conditions in advance.

### <Measurement Conditions of High Performance Liquid Chromatography>

<Tocopherol Phosphate Sodium Salt and dl-α-tocopherol>
Chromatographic column: Shodex (registered trademark of Showa Denko K.K.) F-411

### Temperature: 40° C.

Eluting solution: methanol:water:H₃PO₄=93:6:1
Rate of flow: 1.0 ml/min

### Detection: UV 283 nm

### <Tocopherol Acetate>

Chromatographic column: Shodex (registered trademark of Showa Denko K.K.) F-411

### Temperature: 40° C.

Eluting solution: methanol:acetonitrile=7:3

The eluting solution contained acetic acid (0.02M) and sodium acetate (0.02M).

Rate of flow: 0.7 ml/min

### Detection: UV 283 nm

The wrinkle prevention effects were evaluated by the following evaluation method.

### <Method for Evaluation of Wrinkle Prevention Effects>

Hairless mice were irradiated with UVA/B waves of 300-400 nm ultraviolet light equivalent to or less than 1 MED (minimum erythema dose) at 3 J/cm². Immediately thereafter, each cream 100 μL was applied. These operations were carried out five times a week for twelve weeks. After the UV irradiation and cream application for twelve weeks, wrinkles were visually observed by six scorers. The wrinkle index was scored based on the following criteria to evaluate the wrinkle prevention effects with the average of the six wrinkle indexes.

Wrinkle Index:

1: No wrinkles formed

2: A few wrinkles confirmed

3: Wrinkles confirmed

4: Deep wrinkles confirmed

The results are listed in Table 2.

TABLE 2

| | | | | | Comparative | Comparative | Comparative | Comparative |
|---|---|---|---|---|---|---|---|---|
| | Example 1 | Example 2 | Example 3 | Example 4 | Example 1 | Example 2 | Example 3 | Example 4 |
| | Cream 1 | Cream 2 | Cream 3 | Cream 4 | Cream 5 | Cream 6 | Cream 7 | Cream 8 |
| Property at 40° C. after 24 weeks | No change | No change | No change | No change | Browning | Browning | Browning | Browning |
| | | | | | and | | and | |
| | | | | | separation | | separation | |
| Residual rate of tocopherol/ | 91% | 90% | 90% | 90% | 16% | 12% | 34% | 29% |
| tocopherol derivative | | | | | | | | |
| Wrinkle index | 1.1 | 1.4 | 1.0 | 1.0 | 1.6 | 3.0 | 2.7 | 3.0 |

The results in Table 2 show that the creams in accordance with the present invention have satisfactory wrinkle prevention effects, meaning that the active ingredients are contained in amounts enough to produce wrinkle prevention effects. Moreover, the agents are shown to possess excellent stability after the preparation.

### Examples 5 to 7

Lotions 1 to 3 were prepared based on the composition ratios listed in the following Table 3.

The lotions 1 to 3 were each placed into a sample bottle, which was thereafter sealed. The bottles were introduced in a thermostatic chamber at 40° C., and the lotions were allowed to stand for 24 weeks. After the passage of 24 weeks, the lotions were visually observed to evaluate a change from the state immediately after the lotion was placed in the bottle.

TABLE 3

| | Example 5 | Example 6 | Example 7 |
|---|---|---|---|
| | Lotion 1 | Lotion 2 | Lotion 3 |
| Tocopherol phosphate sodium salt | 2.0 | 2.0 | 2.0 |
| Ascorbic acid-2-phosphate-6- | — | 1.0 | — |
| palmitate trisodium salt | | | |
| Ascorbic acid-2-phosphate-6- | — | — | 1.0 |
| hexyldecanoate trisodium salt | | | |
| Propylene glycol | 6.0 | 6.0 | 6.0 |
| Glycerin | 6.0 | 6.0 | 6.0 |
| Ethanol | 3.8 | 3.8 | 3.8 |
| Phenoxyethanol | 0.1 | 0.1 | 0.1 |
| Dipotassium hydrogenphosphate | 3.2 | 3.2 | 3.2 |
| Tetrasodium | 0.2 | 0.2 | 0.2 |
| ethylenediaminetetraacetate | | | |
| Purified water | 78.7 | 77.7 | 77.7 |

Unit: %

The residual tocopherol in the lotion after the passage of 24 weeks was analyzed by high performance liquid chromatography under the conditions similar to those of Example 1. The residual rate was calculated relative to the tocopherol content in the lotion immediately after the preparation (100) that had been determined under the similar conditions in advance.

The wrinkle prevention effects were evaluated by the evaluation method similar to that of Example 1. The results are listed in Table 4.

TABLE 4

| | Example 5 | Example 6 | Example 7 |
|---|---|---|---|
| | Lotion 1 | Lotion 2 | Lotion 3 |
| Property at 40° C. | Extremely light | Extremely | Extremely |
| after 24 weeks | brown | light brown | light brown |
| Residual rate of tocopherol/ | 95% | 95% | 95% |
| tocopherol derivative | | | |
| Wrinkle index | 1.1 | 1.0 | 1.0 |

The results in Table 4 show that the lotions in accordance with the present invention have satisfactory wrinkle prevention effects, meaning that the active ingredients are contained in amounts enough to produce wrinkle prevention effects. Moreover, the agents are shown to be resistant to discoloration to a certain extent.

What is claimed is:

1. A method of using a composition comprising a tocopherol phosphate and/or a salt thereof as a dermatological anti-wrinkle agent, comprising applying the composition to the skin of a subject in need thereof, wherein the tocopherol phosphate is represented by the following formula (1): 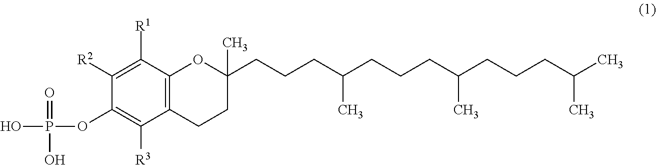
wherein R¹, R², and R³ represent a hydrogen atom or a methyl group independent from each other.

2. The method according to claim 1, wherein the tocopherol phosphate is α-tocopherol phosphate.

3. The method according to claim 2, wherein the tocopherol phosphate is dl-α-tocopherol phosphate.

4. The method according to claim 2, wherein the tocopherol phosphate is d-α-tocopherol phosphate.

5. The method according to claim 1, wherein the tocopherol phosphate is γ-tocopherol phosphate.

6. The method according to claim 5, wherein the tocopherol phosphate is d-γ-tocopherol phosphate.

7. The method according to claim. 1, wherein the tocopherol phosphate is δ-tocopherol phosphate.

8. The method according to claim 7, wherein the tocopherol phosphate is d-δ-tocopherol phosphate.

9. The method according to claim 1, wherein the salt of the tocopherol phosphate is an alkali metal salt of the tocopherol phosphate.

10. The method according to claim 9, wherein the alkali metal salt of the tocopherol phosphate is sodium salt of the tocopherol phosphate.

11. The method according to claim 1, wherein the percentage content of the tocopherol phosphate and/or the salt thereof with respect to the total amount of the composition is in the range of 0.01 to 20 mass %.

12. The method according to claim 1, wherein the composition further comprises an ascorbic acid derivative in addition to the tocopherol phosphate and/or the salt thereof.

13. The method according to claim 12, wherein the ascorbic acid derivative is at least one compound selected from ascorbic acid-2-phosphate, ascorbic acid-2-glucoside, ascorbic acid-6-palmitate, ascorbic acid-2-phosphate-6-palmitate, ascorbic acid-2-phosphate-6-hexyldecanoate, ascorbic acid-2-phosphate-6-stearate, and salts thereof.

14. The method according to claim 12, wherein the percentage content of the ascorbic acid derivative in the composition is in the range of 0.03 to 20 mass %.

15. A method of using a composition comprising a tocopherol phosphate and/or a salt thereof as a cosmetic, comprising applying the composition to the skin of a subject in need thereof, wherein the tocopherol phosphate is represented by the following formula (1): 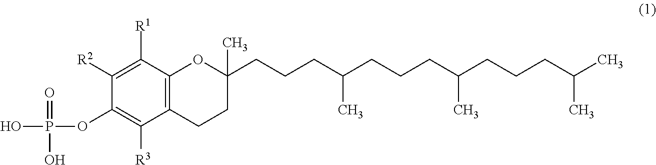
wherein R¹, R², and R³ represent a hydrogen atom or a methyl group independent from each other.
